(19) United States
(12) Patent Application Publication
(10) Pub. No.: US 2023/0014669 A1
(43) Pub. Date: Jan. 19, 2023

(54) WEARABLE ARTICLE, ELECTRONIC MODULE, SYSTEM AND METHOD

(71) Applicant: PREVAYL INNOVATIONS LIMITED, Manchester (GB)

(72) Inventor: Michael John Lynch, Manchester (GB)

(21) Appl. No.: 17/779,483

(22) PCT Filed: Nov. 26, 2020
(86) PCT No.: PCT/GB2020/053009

§ 371 Date: May 24, 2022

(30) Foreign Application Priority Data
Nov. 28, 2019 (GB) 1917346.7

(51) Int. Cl. A61B 5/256; A61B 5/00

(52) U.S. Cl. CPC A61B 5/256; A61B 5/6804; A61B 2560/0223; A61B 2560/028; A61B 2560/0475; A61B 2562/08

(57) ABSTRACT

A wearable article comprising: a first biosensor; an erasable and programmable memory configured to store information relating to the wearable article and/or the first biosensor; and an interface for connection to an electronic module. The interface is configured to permit the transfer of information between the memory and an electronic module connected to the interface. The electronic module is able to read information from the memory and write information to the memory via the interface. The memory may have a single-wire input-output interface. The information may comprise wearable article size information. The wearable article size information may be used to determine a compensation that should be performed to sensor data received from the wearable article to compensate for electrical properties of the wearable article.

### CROSS-REFERENCE TO RELATED APPLICATIONS

This application claims priority from United Kingdom Patent Application number 1917346.7 filed on 28 Nov. 2019, the whole contents of which are incorporated herein by reference.

### BACKGROUND

The present invention is directed towards a wearable article, an electronic module, a system comprising a wearable article and an electronic module, and a method.

Wearable articles can be designed to interface with a wearer of the article, and to determine information such as the wearer's heart rate, rate of respiration, activity level, and body positioning. Such properties can be measured with a sensor assembly that includes a sensor for signal transduction and/or microprocessors for analysis. Wearable articles may be garments which are commonly referred to as ‘smart clothing’ and may also be referred to as ‘biosensing garments’ if they measure biosignals. Typically, different types of wearable articles may have different sensors.

UK Patent Publication No. 2521715 (A) discloses a communication module for personal physical performance monitoring. The module comprises means for mounting to a mounting zone on a sports item. The means for mounting comprises two or more electronic contact terminals for making an electronic contact with the sports item while being mounted thereon. The module additionally comprises a wireless communication unit for communicating with a remote monitoring device, and a processing unit functionally connected to said contact terminals and to said wireless communication unit and capable of processing data received through the contact terminals from sensors in the sports item and/or the wireless communication unit according to data processing instructions. The communication module comprises means for reading an identifier from the sports item while being mounted thereon, and the processing unit is capable of changing said data processing instructions based on the identifier read from the sports item.

It is desirable to provide a system which overcomes at least some of the problems associated with the prior art, whether explicitly discussed herein or otherwise.

### SUMMARY

According to the present disclosure, there is provided a wearable article, an electronic module, a system comprising a wearable article and an electronic module, and a method as set forth in the appended claims. Other features of the invention will be apparent from the dependent claims and the description which follows.

According to a first aspect of the present disclosure, there is provided a wearable article, comprising: a first biosensor; an erasable and programmable non-volatile memory configured to store information relating to the wearable article and/or the first biosensor; and an interface for releasable connection to an electronic module, the interface configured to permit the transfer of information between the memory and an electronic module connected to the interface such that the electronic module is able to read information from the memory and write information to the memory.

Advantageously, the wearable article comprises a biosensor and an erasable and programmable non-volatile memory. The memory is configured to store information relating to the wearable article and/or the first biosensor, but also permits information to be added to the memory and/or updated. In this way, the electronic module is not only permitted to read information from the memory but is also able to write information to the memory. The information written to the memory may relate to usage of the wearable article or testing information for the wearable, for example.

In some implementations, the interface may be configured to permit a wired and/or wireless communicative connection between the electronic module and the memory. The connection may be a releasable mechanical connection. In some implementations, the releasable mechanical connection between the electronic module and the interface may be configured to ensure a suitable configuration of the electronic module and the wearable article for a wireless communicative connection to be established between the electronic module and the memory.

In some implementations, the wearable article may comprise only electronic components associated with the memory, the first biosensor (and optionally other biosensors or sensors) and the interface. In particular, the wearable article may not comprise any further electronic components, for example components associated with power-supply, communications or data processing purposes. In particular, the wearable article may not include any means for communicating data stored in the memory when an electronic module is not connected to the interface. In this way, the electronics within the wearable article can be kept to a minimum. This may reduce failure rates, simplify manufacture and improve wearability. In such implementations, when the electronic module is connected to the interface, the electronic module can obtain information relating to the wearable article from the memory. The electronic module may then transmit the obtained information to another electronic device, for example a server. The electronic module may also be configured to write information to the memory. For example, the electronic module may receive information from a remote device, e.g. a server, and write this information to the memory.

The wearable article comprises a first biosensor (also referred to herein as “biosensing unit”) for measuring biodata/biosignals of the wearer. Here, “biosignal” may refer to any signal in a living being that can be measured and monitored. The term “biosignal” is not limited to electrical signals and can refer to other forms of non-electrical biosignals. A biosensing unit therefore refers to an electronic component that is able to measure a biosignal of the wearer. The biosensing unit may comprise one or more electrodes but is not limited to this arrangement. The biosensing unit may be a textile-based biosensing unit. The terms “biosignal” and “biodata” are used synonymously throughout the specification.

The first biosensor may be a biosensing unit which may be used for measuring one or a combination of bioelectrical, bioimpedance, biochemical, biomechanical, bioacoustics, biooptical or biothermal signals of the wearer. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer's sweat. The biomechanical measurements include blood pressure. The bioacoustics measurements include phonocardiograms (PCG). The biooptical measurements include orthopantomogram (OPG). The biothermal measurements include skin temperature and core body temperature measurements. The biosensing unit may comprise a radar unit. The first biosensor may be a motion sensor. The motion sensor may comprise an accelerometer, and/or gyroscope, and/or magnetometer. The first biosensor may be an inertial measurement unit.

The wearable article may comprise one or more additional sensors. The wearable article may sense one or more signals external to the wearer. The wearable article may comprise any or a combination of a temperature sensor, a camera, a location tracking module such as a GPS module, and a chemical sensor. The wearable article may sense a combination of external signals and biosignals of the wearer.

The electronic module may be in communication with a sensor of the wearable article such as the first biosensor. The electronic module may be configured to wirelessly obtain data from the sensor. For example, the sensor may comprise an RFID component which can be accessed by the electronic module. The electronic module may be conductively connected to the sensor/biosensing unit. The electronic module may be conductively connected to the sensor by a conductor. The conductor may be incorporated into the wearable article. The conductor may be an electrically conductive track or film. The conductor may be a conductive transfer. The conductor may be formed from a fibre or yarn of the textile. This may mean that electrically conductive materials are incorporated into the fibre/yarn.

The releasable mechanical connection may be configured to maintain the electronic module in close proximity to the wearable article. For example, in some implementations, the releasable mechanical connection may be provided by a pocket or the like in the wearable article (particularly a garment). In some implementations, the releasable mechanical connection of the electronic module to the wearable article may be provided by a clip, a plug and socket arrangement, etc. The interface may be configured to maintain the electronic module in a particular orientation with respect to the wearable article when the electronic module is coupled to the wearable article. This may be beneficial in ensuring that the electronic module is securely held in place with respect to the wearable article and/or that any electronic or communicative coupling of the electronic module and the wearable article (or a component of the wearable article) can be optimized. The mechanical coupling may be maintained using friction or using a positively engaging mechanism, for example. The interface may be configured to permit a pin and socket connection to the electronic module. In some examples, the connection may be formed using spring pins. In other examples, a magnetic connection may be formed.

The releasable electronic module may contain all of the components required for data transmission and processing such that the wearable article only comprises sensors (e.g. biosensors) and a memory. In this way, manufacture of the wearable article may be simplified. Furthermore, since the electronic module may directly transmit information to a server via a wireless communications network, it may be possible to remove the need for a mobile telephone in a garment control system. This may have the advantage of simplifying a smart clothing system. It may also have the advantage of reducing costs associated with a smart clothing system and/or of increasing usability of a smart clothing system. For example, since the electronic module of the present invention may not require all of the functionality of a mobile telephone (such as, for example, a relatively large touchscreen and/or voice telephony functionality), the electronic module may be made smaller than a typical mobile telephone or “smart phone”. This may have the effect of reducing overall weight of the system and/or reducing bulkiness of the electronics. As a result, user experience may be improved. In addition, it may be easier to clean a wearable article which has fewer electronic components attached thereto or incorporated therein. Furthermore, the removable electronic module may be easier to maintain and/or troubleshoot than embedded electronics.

The memory may be an erasable programmable read-only memory (EPROM), an electrically erasable programmable read-only memory (EEPROM) or a floating-gate memory such as flash memory. Advantages of using an EEPROM memory may include the ability to work reliably in a relatively high impedance signal line and requiring a relatively low power input compared to other memory types.

In some implementations, the memory may be connected to the interface using a bidirectional line which permits signals to be read from and written to the memory. The bidirectional line may be a single-wire bidirectional line which may, in particular examples, be a one-wire bus. The wearable article may therefore comprise a (single-wire) bidirectional line that connects the interface to the memory. Where the memory is connected via the single-wire bidirectional line, the wearable article may further comprise at least one sensor connected to the single-wire bidirectional line. By using a single-wire bidirectional line, the number of connections required to transfer information between the memory and the electronic module may be minimized, which may assist in simplifying manufacture, reducing failure and/or reducing a size of the interface between the memory and the electronic module, as well as reducing a size of the electronic module itself. It is appreciated that even with a single-wire protocol, a separate ground line may still be provided. The present disclosure is not limited to single-wire input-output interfaces and single-wire bidirectional lines although particular advantages are achieved in these examples. Two-wire bidirectional lines, three-wire bidirectional lines or four or more wire bidirectional lines may also be used in some examples. The bidirectional lines may use any existing serial protocol such as Serial Peripheral Interface (SPI), Inter-Integrated Circuit (I2C), Controller Area Network (CAN), Recommended Standard 232 (RS-232), and 1-wire

It may be desirable to use as few connections as possible to enable the transfer of information between the electronic module and the memory, and optionally the first biosensor. In particular, by providing as few connections as possible between the interface and the electronic module, manufacture may be simplified, the electronic components in the wearable article may be kept as unobtrusive as possible and failures may be reduced. In some implementations, the electronic module may additionally or alternatively be configured to wirelessly obtain information from the first biosensor, for example by means of RFID technology or other means of wireless communication known to the person skilled in the art.

The information stored in the memory may comprise at least one of: an identifier associated with the wearable article, a wearable article type, a wearable article size, a wearable article colour, an identifier associated with the first biosensor, a type of the first biosensor, an electrical property associated with the first biosensor, calibration data associated with the first biosensor, a user identifier and usage information.

Storing the wearable article (e.g. garment) size may be particularly beneficial since the size of the wearable article may relate to the electrical properties (such as impedance, capacitance, tolerance, etc.) of the electrodes embedded within the wearable article. A physically larger wearable article may have a different impedance to a smaller wearable article to the different length of the conductive traces within the wearable article. The electronic module may read the memory to obtain the size of the wearable article and then determine a compensation that should be performed to take into account the impedance and tolerance of the electrodes. This is helpful when the electronic module has no access to a server associated with the wearable article. For example, in some operations, a unique ID associated with the wearable article may be sent to a server and the server would perform a lookup and respond with the corresponding configuration data for the wearable article. However, if such a connection to the server were not possible, having this information stored in the memory of the wearable article would be useful.

The memory may store electronics information for the wearable article. The electronics information may indicate the electrical properties of the wearable article and particularly of any electrodes of the wearable article. The electronics information may include calibration information which may be written to the memory during testing. In a particular example, the electronics information comprises the impedance of the electrodes of the wearable article. In a particular example, the electronics information comprises an electrode identifier. The compensation to be applied may be determined based on the wearable article size and the electronics information. For example, wearable articles of different sizes (e.g. small, medium, and large) may comprise the same type of electronics but due to the different fabric stretch properties of these different wearable articles the electronics may perform differently. Using the combination of electrical (e.g. impedance) and stretch properties, the electronic module is able to determine a compensation to apply. In some examples, certain sizes of wearable article (e.g. extra small, small, and medium) may have first type of electronics, while other sizes of wearable article (e.g. large, extra large, and extra extra) may have a different, second, type of electronics. Storing the electronics information and the wearable article size information in the memory means that the electronic module is able to compensate for these variations when processing sensor data obtained from the wearable article.

Storing different types of information in the memory provides additional benefits particular in terms of determining a point of failure during manufacturing, storing, and/or transporting. For example, a particular type of wearable article, a particular size of wearable article, a particular manufacturer of electronics or wearable articles, a particular factory or even a particular operator may be responsible for failure in the wearable articles. Storing identifying information in the memory of the wearable article enables the present disclosure to rapidly and easily determine which of these factors (or other factors) is responsible for the failure. For example, the memory of a plurality of wearable articles which have malfunctioned may be read to determine whether they share any common identifying information in their memory.

Another benefit associated with storing different types of information in the memory is in terms of identifying whether the wearable article or a component of the wearable article is counterfeit. The electronic module may read identifying information from the memory such as an identifier for the wearable article or an electronics component of the wearable article (e.g. an electrode) and compare the same to a database. The database may be maintained on a server.

The information stored on the memory may be encrypted. This helps ensure data security especially as the erasable and programmable memory may not provide write protection. Encrypting the information may prevent or make it more difficult to clone wearable articles.

The memory may be configured to store information relating to quality control or quality assurance. In particular, the information relating to quality control or quality assurance may be temporal information. In some implementations, the memory may be configured to store a plurality of items of temporal information relating to quality control or quality assurance. The information may comprise an operator ID and/or a test rig ID.

In particular, during manufacture of a smart wearable article (e.g. smart clothing), a subassembly comprising electronic components (e.g. sensors, electrodes, memory) may be combined with a wearable article to form the smart wearable article. In order to ensure that the electronic components meet a high quality standard before, during and after manufacture of the smart wearable article, quality control and/or quality assurance procedures may be followed. For example, at least one electronic component associated with the smart wearable article may be tested to ensure that a quality threshold is met. Such testing may be performed more than once during the manufacturing process.

As an example, a sensor electrode may be provided on a fabric subassembly for integration into a garment. The sensor electrode may be tested after production of the sensor electrode. If the sensor electrode is manufactured at a different location to the rest of the garment, the sensor electrode may be retested on arrival at the garment manufacture location. The sensor electrode may then be tested again after integration into the garment. In this way, it is possible to ensure that any problems found with the sensor electrode can be linked to the source of the problem (e.g. sensor manufacture, sensor storage or garment manufacture). In addition, it is possible to ensure that QA/QC procedures are followed.

If a sensor electrode is tested and found to be faulty before integration into a garment, for example directly after the sensor electrode is manufactured, this can improve efficiency since the faulty sensor electrode has been identified at an early stage and will not be integrated into a garment. If the faulty sensor electrode was only tested after integration, this could lead to waste of time and resources used to integrate the faulty sensor electrode into a garment only then to find that the component was faulty and could not be used.

Furthermore, pre-fabricated sensor assemblies may be stored for some time before being integrated into garments. It may therefore be beneficial to retest the sensor electrode before integration into a garment in order to ensure that the storage has had no adverse effect on the sensor electrode. It may also be possible to detect trends in the data which may indicate an ongoing storage issue. For example, a high failure rate after storage may indicate that the sensor assemblies are being stored at too high a temperature or humidity level. By testing the sensor electrodes and monitoring the trends, it may be possible to identify and remedy such problems.

At each testing stage, information may be written to the memory to indicate that a particular QC/QA step has been performed. The information may comprise a time or date stamp. The information may also comprise information relating to the test performed, including what the test was, where it was performed and by whom. This information may be stored in the memory for later retrieval. It may then be possible to verify at a later time whether or not the QA/QC procedures were correctly followed during manufacture.

Although sensor electrodes have been referred to above, it will of course be appreciated that other components present on the fabric subassembly or in the garment (or other wearable article) can also be tested in this manner. For example, the memory, the interface, conductive traces and/or connections between components may also be tested and QA/QC data relating to these components may be stored in the memory.

A test rig may be used to test the function of a completed smart wearable article. For example, the integrity of the sensor connections in the wearable article may be tested. The results of the testing may be written to the memory, for example a programmable memory. The test rig may also write wearable article information to the memory. The wearable article information may include a wearable article type, size, colour, identifier, available sensors, tolerance of electrodes present in the wearable article, tolerance of sensors present in the wearable article, an electrical property associated with a component of the wearable article, manufacturing data and/or an operator ID.

The wearable article information may be obtained by the test rig scanning a machine-readable code on the wearable article. The machine-readable code may be a visual code such as a barcode or a quick-response (QR) code or an augmented-reality (AR) marker embedded within the fabric or otherwise incorporated onto the fabric. The data from the test rig may also be sent to a server associated with the wearable article. When the electronic module is later connected to the wearable article, it can read the memory and check that the wearable article was tested successfully (e.g. determine that the appropriate date/time stamp or stamps are present). The electronic module may then determine which sensors are available based on the information obtained from the memory. The electronic module may then configure itself to obtain only data for these sensors and transmit the same.

However, if a wearable article memory is not programmed during manufacture, the programmable memory of each wearable article will have a unique identifier which will allow the memory (and hence the wearable article) to be uniquely identified. The wearable article information may be populated in the memory by the electronic module, for example by the electronic module obtaining the relevant information from the server and writing it to the wearable article memory.

The wearable article may comprise a machine-readable code. In some implementations, the machine-readable code encodes at least a portion of the information stored in the memory. The machine-readable code may comprise a visual code and may comprise at least one of: a barcode, a quick-response (QR) code and an augmented-reality (AR) marker.

In general, the wearable article may comprise a visual symbol which comprises, encoded therein, a unique code string that identifies the wearable article. An electronic device comprising a camera may image the visual symbol and transmit information representing the visual symbol. The information may be in the form of a data string obtained from the image. The data string may be a simple digitised representation of the visual symbol or may be an encrypted version of the code string. The method may run a decoding algorithm to generate the code string from the data string.

The machine-readable code may also be used for motion tracking. In some implementations, the machine-readable code may be a marker may be located on (i.e. readable from) an outside surface of the wearable article. The at least one marker may comprise a code string identifying the wearable article encoded into a visual symbol. The marker may be a 2D image. The marker may be a fiducial marker optionally in the form of a 2D image. The marker may be an Augmented Reality (AR) marker with additional information in the form of the code string encoded therein. In some implementations where the machine-readable code is an AR marker, the marker may cause a particular graphic or media to be displayed on a mobile telephone or other electronic device when the mobile telephone or electronic device scans the marker. For example, the marker may be associated with a particular body part and the graphic or media excerpt which is caused to be displayed on the mobile telephone may be associated with the particular body part.

The marker may comprise a plurality of markers. The plurality of markers may be located at different locations on the wearable article. The plurality of markers may be arranged in a geometric pattern. The plurality of markers may be arranged together on the wearable article to form a decorative item. The plurality of markers may be located at different locations on the wearable article. The marker may be integrated into the wearable article. The marker may be printed onto the wearable article. Any known printing technique may be used such as screen printing or inkjet printing. The marker may be incorporated into the stitching of the wearable article (e.g. a garment), and/or a seam of the garment, and/or a hem of the garment, and/or a neckline of the garment, and/or a collar of the garment, and/or a sleeve of the garment, and/or a cuff of the garment, and/or a pocket of the garment, and/or a body of the garment, and/or a fastener of the garment. The fastener may be a zipper, button, clasp, toggle, stud, snap fastener, popper, eyelet, buckle, tie or ribbon.

In some examples, the marker has a limited visual footprint on the wearable article. This means that the marker is sufficiently small that it is not easily visible by the naked eye but is still visible in the image captured by the image capturing device. In this way, the marker does not affect or has a minimal effect on the appearance of the wearable article. In some examples, the marker is visible to the naked eye. The marker may be incorporated into or form part of visual element on the wearable article which may be a decorative item in the wearable article. The decorative item may be a logo, design, image or pattern on the wearable article. In this way, the marker may contribute to or enhance the appearance of the wearable article.

The memory may store information relating to how many times an electronic module has been connected to the wearable article. The memory may comprise a counter which can be incremented each time an electronic module is connected to the wearable article. That is, when an electronic module is connected to the interface, the wearable article is configured to increment the counter stored in the memory. This incrementing of the counter may be performed in response to an instruction received from the electronic module. Incrementing the counter may provide information relating to frequency of use of the wearable article and/or of the electronic module. Usage information may be used to calculate a level of wear or degradation of the electronics present in the wearable article and to offset any potential errors. For example, an electrical property of a component in the wearable article, e.g. a resistance or an impedance, may change over time as the wearable is worn and washed. The information relating to number of uses could be helpful to determine whether any offset should be applied to data obtained in order to compensate for a known degradation rate. Any degradation rate may be determined through batch testing or simulation prior to mass manufacture, for example. In some implementations, a mathematical model may be generated to approximate wash cycles. Electrodes or other electronic components in the wearable article may deteriorate over the course of 25 wash cycles at ISO6330, for example, regardless of construction. By obtaining usage data from the memory and/or data from a counter indicating the number of times an electronic module has been connected to the wearable, the electronic module is able to use known degradation information to apply an offset value or correction factor and thereby improve the accuracy of data collection over the lifetime of the wearable article.

The wearable article may further comprise a second biosensor. It will be appreciated that features mentioned above with regard to the first biosensor may apply equally to the second biosensor. The first biosensor and the second biosensor may be of different types or of the same type.

The wearable article may further comprise a textile material such as a fabric material. The memory, interface and/or biosensor may be integrated or otherwise incorporated into the textile material. The wearable article may be a garment. The garment may refer to an item of clothing or apparel. The garment may be a top. The top may be a shirt, t-shirt, blouse, sweater, jacket/coat, or vest. The garment may be a dress, brassiere, shorts, trousers (pants), arm or leg sleeve, glove, armband, underwear, headband, hat/cap, collar, wristband, stocking, sock, or shoe (footwear), athletic clothing, swimwear, wetsuit or drysuit. The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the wearable article/garment. Polyester, polycotton, nylon and viscose are synthetic fibres that may be used in the garment.

It may be desirable to avoid direct contact of the electronic module with the wearer's skin while the wearable article is being worn. In particular, it may be desirable to avoid the electronic module coming into contact with sweat or moisture on the wearer's skin. The electronic module may be provided with a waterproof coating or waterproof casing. For example, the electronic module may be provided with a silicone casing. It may further be desirable to provide a pouch or pocket in the wearable article to contain the electronic module in order to prevent chafing or rubbing and thereby improve comfort for the wearer. The pouch or pocket may be provided with a waterproof lining in order to prevent the electronic module from coming into contact with moisture.

According to a second aspect of the present disclosure, there is provided an electronic module configured to be releasably connected (e.g. mechanically) to an interface of a wearable article, the electronic module configured to obtain information from an erasable and programmable memory of the wearable article and to write information to the memory of the wearable article when the electronic module is connected to the interface of the wearable article. The wearable article may be the wearable article of the first aspect of the present disclosure.

The electronic module may be further configured to be communicatively connected to the interface in a wired and/or wireless manner.

The electronic module may be releasably connected to all manner of wearable articles. Not all of the wearable articles will comprise the same sensors. Some wearable articles may comprise only one biosensor; other may comprise a plurality of biosensors. It may be desirable for the electronic module to be able to operate with all kinds of sensors or at least as many sensors as possible.

The electronic module may comprise a plurality of submodules and be configured to selectively enable and/or disable at least one submodule based on information obtained from the memory. In particular, each submodule may be configured to interact with a sensor of a particular type. The electronic module may be configured to selectively enable at least one of the submodules in order to ensure that submodules which are relevant to the sensors present on the wearable article to which the electronic module is connected are active. The electronic module may also be configured to selectively disable at least one of the submodules if it is determined that the particular submodule to be disabled is not relevant to any of the sensors present on the particular wearable article to which the electronic module is connected. In this way, it may be possible to conserve power.

The electronic module may be configured to transmit information to a server and/or to write information from the server to the memory. The electronic module may be configured to configure a data stream based on information obtained from the memory. In particular, the electronic module may obtain information from the memory that indicates which sensor or sensors are present in the wearable article. In addition, the electronic module may configure a data stream such that only information relating to the appropriate sensor is transmitted as part of the data stream. In this way, it is possible to avoid noise from the other sensors and/or submodules polluting the data stream. In addition, the data stream may use less bandwidth and the transmission may require less power.

The electronic module may be further configured to connect to the interface of the wearable article using a pin and socket connection. For example, the electronic module may comprise a plurality of pins configured to engage with sockets of the interface, or vice versa. It will be appreciated, however, that other connection means may be provided. In some implementations, the connections may be formed by spring pins. In other implementations, a magnetic connection between the electronic module and the interface may be provided.

The electronic module may further comprise an input-output interface for connection to the memory associated with the wearable article. The input-output interface may be a single-wire input-output interface. In this way, a number of connections to the interface may be minimized.

The electronic module may comprise flexible electronics such as a flexible printed circuit (FPC). The electronic module may be configured to be electrically coupled to the wearable article.

In some implementations, the electronic module may also be configured to access the memory associated with the wearable article via a wireless connection. For example, the electronic module may be configured to wirelessly access an RFID tag associated with the wearable article.

Beneficially, the electronic module may make it possible to use a single electronic module with a plurality of wearable articles. It will be appreciated that the wearable articles may be of the same type or of a variety of types. In this way, manufacturing is simplified and costs may be reduced. It will be appreciated that the features described herein with reference to a single wearable article may also be applied to each wearable article where the removable electronic module is used with multiple wearable articles.

The electronic module may be operable to communicate data wirelessly via one or more base stations. For example, the electronic module may be able to communicate via one or more wireless communication protocols such as used for communication on: a wireless wide area network (WWAN), a wireless metroarea network (WMAN), a wireless local area network (WLAN), a wireless personal area network (WPAN), a near field communication (NFC), and a cellular communication network. The cellular communication network may be a fourth generation (4G) LTE, LTE Advanced (LTE-A), fifth generation (5G), sixth generation (6G), and/or any other present or future developed cellular wireless network. The electronic module may be able to communicate via short-range local communication over WLAN, WPAN, NFC, or Bluetooth WiFi or any other electromagnetic RF communication protocol.

The electronic module may comprise a power source or a plurality of power sources. The power source may be conductively connected to the wearable article by a conductor. The conductor may be a conductive transfer. The conductor may be formed from a fibre or yarn of the garment. This may mean that an electrically conductive material such as graphene is incorporated into the fibre/yarn. The power source may be a battery. The battery may be a rechargeable battery. The battery may be a rechargeable battery adapted to be charged wirelessly such as by inductive charging. The power source may comprise an energy harvesting device. The energy harvesting device may be configured to generate electric power signals in response to kinetic events such as kinetic events performed by a wearer of the garment. The kinetic event could include walking, running, exercising or respiration of the wearer. The energy harvesting material may comprise a piezoelectric material which generates electricity in response to mechanical deformation of the converter. The energy harvesting device may harvest energy from body heat of a wearer of the wearable article. The energy harvesting device may be a thermoelectric energy harvesting device.

The electronic module may be configured to perform a testing function on the wearable article and write a result of the testing function to the memory of the wearable article. The electronic module may be further configured to write wearable article information to the memory of the wearable article.

The electronic module may be a component of a test rig. That is, there may be provided a test rig. The test rig may comprise the electronic module as disclosed herein. The test rig may comprise a reader. The reader may be arranged to read a machine-readable code on the wearable article. The machine-readable code may comprise at least a component of the wearable article information.

According to a third aspect of the present disclosure, there is provided a system comprising a first wearable article as disclosed herein and an electronic module as disclosed herein. In some implementations, the system may further comprise a second wearable article as disclosed herein. In some implementations, the first wearable article and the second wearable article may be of different types. In systems comprising a plurality of wearable articles, the electronic module may be configured to be releasably mechanically connected to each of the wearable articles in the system.

According to a fourth aspect of the present disclosure, there is provided a method of operating an electronic module, the method comprising: connecting the electronic module to an interface of a wearable article; obtaining, by the electronic module, information from an erasable and programmable memory of the wearable article; and writing, by the electronic module, information to the memory of the wearable article.

In some implementations, the method may further comprise configuring, by the electronic module, a data stream based on information obtained from the memory.

In some implementations, the electronic module may comprise a plurality of submodules and the method may further comprise selectively enabling and/or disabling at least one submodule based on information obtained from the memory.

In some implementations, the method may further comprise transmitting information from the memory to a server and/or write information from the server to the memory.

In some implementations, the method may further comprise connecting the electronic module to the interface of the wearable article using a pin and socket connection.

In some implementations, the method may further comprise connecting the electronic module to the memory associated with the wearable article via a single-wire input-output interface.

In some implementations, the method may further comprise establishing a wired or wireless communicative connection between the electronic module and the memory.

According to a fifth aspect of the present disclosure, there is provided a wearable article. The wearable article comprises a memory configured to store information relating to the wearable article. The memory comprises a single-wire input-output interface. The wearable article comprises at least one sensor comprising a single-wire input-output interface. The wearable article comprises an interface for releasable mechanical connection to an electronic module. The wearable article comprises a single-wire bidirectional line connecting the single-wire input-output interface of the memory to the single-wire input-output interface of the at least one sensor and the interface. The interface is configured to permit the transfer of information between the memory, the at least one sensor and an electronic module connected to the interface via the single-wire bidirectional line.

Advantageously, the wearable article comprises a memory and a sensor that use the same shared single-wire bidirectional line. This reduces the number of communication lines in the wearable.

The wearable article may comprise some or all of the features of the wearable article of the first aspect of the disclosure.

According to a sixth aspect of the present disclosure, there is provided a system. The system comprises a wearable article of the fifth aspect of the present disclosure. The system comprises an electronic module configured to be releasably connected to an interface of a wearable article. The electronic module is configured to obtain information from the memory of the wearable article and/or to write information to the memory of the wearable article when the electronic module is connected to the interface of the wearable article.

The electronic module may comprise some or all of the features of the electronic module of the second aspect of the disclosure.

According to a seventh aspect of the present disclosure, there is provided an electronic module configured to be releasably mechanically connected to an interface of a wearable article. The electronic module is configured to obtain wearable article size information from a memory of the wearable article, and is further configured to a determine a compensation that should be performed to sensor data received from the wearable article to compensate for electrical properties of the wearable article using the wearable article size information.

The electronic module may comprise some or all of the features of the electronic module of the second aspect of the disclosure.

According to a eighth aspect of the present disclosure, there is provided a wearable article comprising: a first biosensor; a memory configured to store wearable article size information; an interface for releasable mechanical connection to an electronic module, wherein the interface is configured to permit the transfer of the wearable article size information from the memory to an electronic module connected to the interface.

The wearable article may comprise some or all of the features of the wearable article of the first or fifth aspect of the disclosure.

It will of course be appreciated that any of the features disclosed above in connection with an aspect of the invention may be combined with the features disclosed in connection with any other aspect of the invention without departing from the scope of the invention

### BRIEF DESCRIPTION OF THE DRAWINGS

Examples of the present disclosure will now be described with reference to the accompanying drawings, in which:

FIG. 1 shows a schematic view of a system according to aspects of the present disclosure;

FIG. 2 shows a schematic view of another system according to aspects of the present disclosure;

FIG. 3 shows a schematic view of an electronic module according to aspects of the present disclosure;

FIG. 4 shows a schematic view of another electronic module according to aspects of the present disclosure; and

FIG. 5 shows a schematic view of a single-wire bidirectional line arrangement according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of electronic device which may be worn by a user such as a smart watch, necklace, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, brassiere, shorts, pants, arm or leg sleeve, vest, jacket/coat, glove, armband, underwear, headband, hat/cap, collar, wristband, stocking, sock, or shoe, athletic clothing, swimwear, wetsuit or drysuit. The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the particular application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment. The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment.

The following description refers to particular examples of the present disclosure where the wearable article is a garment. It will be appreciated that the present disclosure is not limited to garments and other forms of wearable article are within the scope of the present disclosure as outlined above.

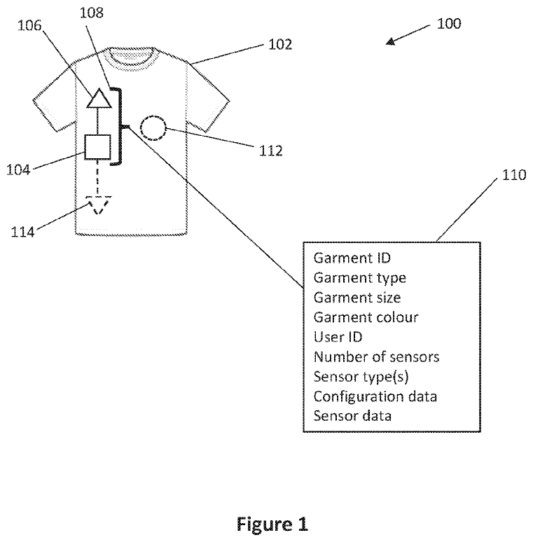

Referring to FIG. 1, there is shown an example system 100 according to aspects of the present invention. The system 100 comprises a garment 102 and electronic module 112. The garment 102 comprises a memory 104 and a first biosensor 106. The garment 102 also comprises an interface 108 which is configured to permit the transfer of information 110 between the memory 104 and the electronic module 112 which can be releasably mechanically connected to the interface 108. The interface 108 may also be configured to permit the transfer of information between the first biosensor 106 and the electronic module 112. The interface 108 may be configured to permit wired and/or wireless communicative connection of the electronic module 112 to the garment 102.

In some implementations, the releasable mechanical connection between the electronic module 112 and the interface 108 may be configured to ensure a suitable configuration of the electronic module 112 and the garment 102 for a wireless communicative connection to be established between the electronic module 112 and the memory 104.

In some implementations, the garment 102 may comprise only electronic components associated with the memory 104, the first biosensor 106 (and other sensors/biosensors) and the interface 108. In particular, the garment 102 may not comprise any further electronic components, for example components associated with power-supply, communications or data-processing purposes. In particular, the garment 102 may not include any means for communicating data stored in the memory when an electronic module 112 is not connected to the interface. In this way, the electronics within the garment 102 can be kept to a minimum. This may reduce failure rates, simplify manufacture and improve wearability of the garment. In such implementations, when the electronic module 112 is connected to the interface 108, the electronic module 112 can obtain information 110 relating to the garment 102 from the memory 104. The electronic module 112 may transmit the obtained information 110 to another electronic device, for example a server. The electronic module 112 may also be configured to write information to the memory 104. For example, the electronic module 112 may receive information from a remote device, e.g. a server, and write this information to the memory 104.

The garment 102 is a T-shirt, but may be an item of clothing or apparel. For example, the garment may be a top such as a shirt, t-shirt, blouse, sweater, jacket/coat, or vest. In other implementations, the garment may be a dress, brassiere, shorts, trousers (pants), arm or leg sleeve, glove, armband, underwear, headband, hat/cap, collar, wristband, stocking, sock, or shoe, athletic clothing, swimwear, wetsuit or drysuit.

The memory 104 may be a programmable memory. In some implementations, the memory 104 may be an EEPROM memory. Advantages of using an EEPROM memory may include the ability to work reliably in a relatively high impedance signal line and requiring a relatively low power input compared to other memory types.

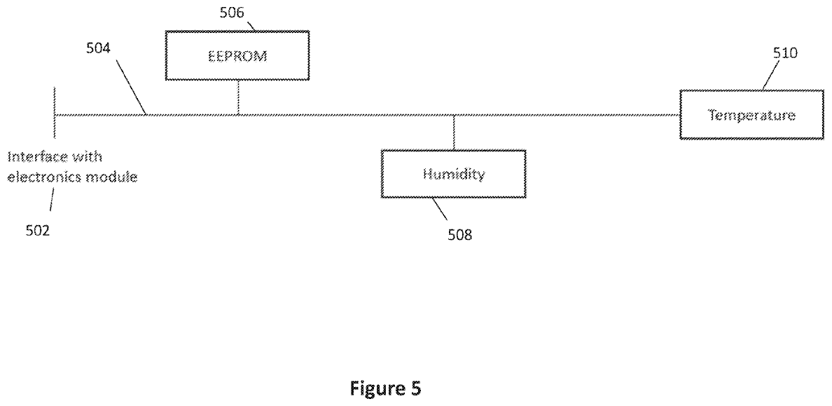

In some implementations, the memory 104 may be connected via a bidirectional line such as a single-wire bidirectional line. In this case, the memory 104 may only require one data wire and one ground wire to connect the memory 104 to the interface 108 and, as a result, to the electronic module 112 when the electronic module 112 is connected to the interface 108. By using a single-wire bidirectional line, the number of connections required to transfer information 110 between the memory 104 and the electronic module 112 may be minimized, which may assist in simplifying manufacture, reducing failure and/or reducing a size of the interface 108 between the memory 104 and the electronic module 112, as well as reducing a size of the electronic module 112 itself. Further advantages of a single-wire bidirectional line are described below with reference to FIG. 5.

The first biosensor 106 may be used for measuring one or a combination of bioelectrical, bioimpedance, biochemical, biomechanical, bioacoustics, biooptical or biothermal signals of the wearer of the garment 102. In some embodiments, the garment 102 may also comprise a second biosensor 114 in addition to the first biosensor 106. The interface 108 may be configured to permit the transfer of information between the second biosensor 114 and the electronic module 112. The first and second biosensors 106, 114 may be of the same type or may be of different types. It will of course be appreciated that any number of biosensors, which may be of the same type or of different types, may be provided according to requirements.

The electronic module 112 is configured to be releasably mechanically connected to the garment 102 via the interface 108. The interface 108 may be configured to permit a wired and/or wireless communicative connection between the electronic module 112 and the garment 102. In some implementations, the interface 108 may be configured to permit an electrical connection such as a pin and socket connection of the electronic module 112 to the garment 102. In some implementations, the interface 108 may comprise a plurality of sockets with which a plurality of pins of the electronic module 112 may engage. Alternatively, the interface 108 may comprise a plurality of pins which are configured to engage with corresponding sockets in the electronic module 112. In some implementations, the connectors may be spring pins. In other implementations, a magnetic connection between the electronic module 112 and the interface 108 may be provided.

It may be desirable to use as few connections as possible to enable the transfer of information 110 between the electronic module 112 and the memory 104, and optionally the first biosensor 106. In particular, by providing as few connections as possible between the interface 108 and the electronic module 112, manufacture may be simplified, the electronic components in the garment may be kept as unobtrusive as possible and failures may be reduced.

In implementations where the interface 108 also enables the transfer of information 110 between the first biosensor 106 and the electronic module 112, a plurality of connections from the biosensor 106 to the interface 108 and from the interface 108 to the electronic module 112 may be required. In some implementations, three pin/socket connections may be provided for allowing the transfer of information between the first biosensor 106 and the electronic module 112 via the interface 108. It will be appreciated of course that more or fewer connections may be provided according to requirements. Here, too, it may be desirable to provide as few connections as possible in order to render the interface 108 as unobtrusive as possible. It will further be appreciated that other types of connections other than pin/socket may be provided.

In some implementations, the electronic module 112 may additionally or alternatively be configured to wirelessly obtain information 110 from the first biosensor 106 (and/or the second biosensor 114 where present), for example by means of RFID technology or other means of wireless communication known to the person skilled in the art.

The information 110 which is stored in the memory 104 and which may be transferred via the interface 108 may include at least one of: an identifier associated with the garment 102, a garment type, a garment size, a garment colour, an identifier associated with the first biosensor 106, a type of the first biosensor 106, an electrical property associated with the first biosensor 106, calibration data associated with the first biosensor 106, an identifier associated with the second biosensor 114, a type of the second biosensor 114, an electrical property associated with the second biosensor 114, calibration data associated with the second biosensor 114, a user identifier and usage information. It will be appreciated that the information 110 illustrated in FIG. 1 is by way of example only. In particular, not all of the illustrated types of information may be present. Likewise, further types of information which are not specified in FIG. 1 may be present.

Storing the garment size may be particularly beneficial since the size of the garment 102 relates to the impedance and tolerance of the electrodes embedded within the garment. A physically larger garment will have a different impedance to a smaller garment (with the same electrodes) due to the different length of the conductive traces within the garment. The electronic module 112 may read the memory 104 to obtain the size of the garment 102 and then determine a compensation that should be performed to take into account the impedance and tolerance of the electrodes. This is helpful when the electronic module 112 has no access to a server associated with the garment. For example, in some operations, a unique ID associated with the garment 102 may be sent to a server and the server would perform a lookup and respond with the corresponding configuration data for the garment 102. However, if such a connection to the server were not possible, having this information stored in the memory 104 of the garment 102 would be useful.

The memory 104 may store information relating to how many times an electronic module has been connected to the garment 102. For example, the memory 104 may comprise a counter which can be incremented each time the electronic module 112 is connected to the garment 102. This may provide information relating to frequency of use of the garment 102 and/or of the electronic module 112. Usage information may be used to calculate a level of wear or degradation of the electronics present in the garment and to offset any potential errors. For example, an electrical property of a component in the garment, e.g. a resistance or an impedance, may change over time as the garment is worn and washed. The information relating to number of uses could be helpful to determine whether any offset should be applied to data obtained in order to compensate for a known degradation rate. Any degradation rate may be determined through batch testing or simulation prior to mass manufacture, for example. In some implementations, a mathematical model may be generated to approximate wash cycles. Electrodes or other electronic components in the garment may deteriorate over the course of 25 wash cycles at ISO6330, for example, regardless of construction. By obtaining usage data from the memory and/or data from a counter indicating the number of times an electronic module has been connected to the garment, it may be possible to use known degradation information to apply an offset value or correction factor and thereby improve the accuracy of data collection over the lifetime of the garment.

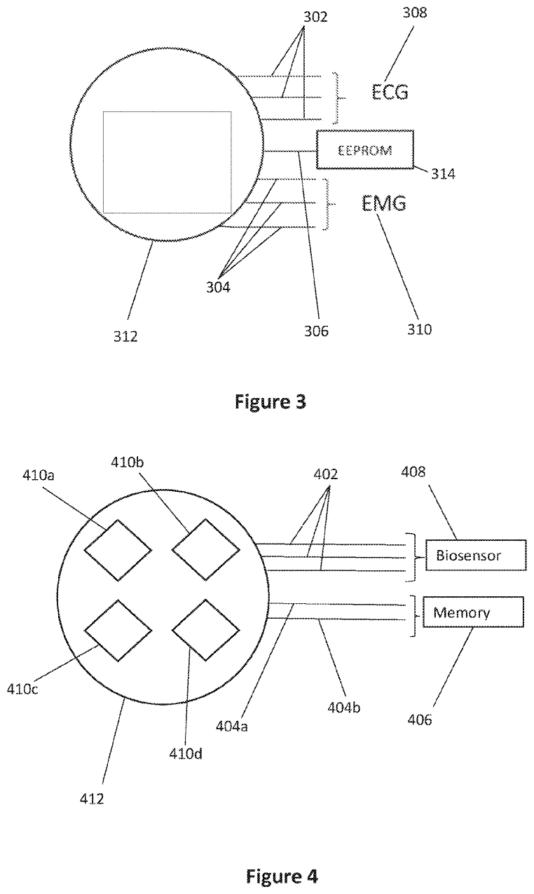

The interface 108 may be configured to maintain the electronic module 112 in a particular orientation with respect to the garment 102 when the electronic module 112 is coupled to the garment 102. This may be beneficial in ensuring that the electronic module 112 is securely held in place with respect to the garment 102 and/or that an electronic or communicative coupling of the electronic module 112 and the garment 102 (or a component of the garment 102) can be optimized. In some embodiments, a mechanical connection or coupling between the interface 108 and the electronic module 112 may be maintained using friction or using a positively engaging mechanism, for example. In some embodiments, the releasable mechanical coupling may be provided by a pocket in the garment 102. For example, the electronic module 112 may be placed into a pocket provided on the garment 102 which is configured to allow a wireless communicative connection to be established between the electronic module 112 and the memory 104. For example, the pocket may be configured such that the electronic module 112 is located in close proximity with the memory 104 in order to facilitate wireless data transfer. It will be appreciated, however, that any suitable mechanical coupling may alternatively or additionally be used. The interface 108 is described in more detail below with reference to FIGS. 3, 4 and 5.

The memory 104 may be configured to store information relating to quality control or quality assurance, for example during manufacture of the garment. In some implementations, the information relating to quality control (QC) or quality assurance (QA) may be temporal information. For example, the information relating to QC or QA may be a time and/or date stamp indicating when a QC or QA check was carried out during the manufacturing process of the garment 102.

During manufacture of smart clothing, a fabric subassembly comprising electronic components (e.g. sensors, electrodes, memory) may be combined with a garment to form a smart garment. In order to ensure that the electronic components meet a high quality standard before, during and after manufacture of the smart garment, quality control and/or quality assurance procedures may be followed. For example, at least one electronic component associated with the smart garment may be tested to ensure that a quality threshold is met. Such testing may be performed more than once during the manufacturing process.

As an example, a sensor electrode may be provided on a fabric subassembly, which also comprises a memory, for integration into a garment. Such integration may be performed using heat transfer, sewing, fabric welding or other integration techniques known to the skilled person. Fabric subassembly may not be required in all examples of the present disclosure as the sensor electrode (and other electronics components such as the memory) may be directly integrated into the garment. The garment may be manufactured in one-piece such as by using knitting techniques, and the electronic components may be integrated into the garment directly. The sensor electrode may be tested after production of the sensor electrode. If the sensor electrode is manufactured at a different location to the rest of the garment, the sensor electrode may be retested on arrival at the garment manufacture location. The sensor electrode may then be tested again after integration into the garment. In this way, it is possible to ensure that any problems found with the sensor electrode can be linked to the source of the problem (e.g. sensor manufacture, sensor storage or garment manufacture). In addition, it is possible to ensure that QA/QC procedures are followed.

If a sensor electrode is tested and found to be faulty before integration into a garment, for example directly after the sensor electrode is manufactured, this can improve efficiency since the faulty sensor electrode has been identified at an early stage and will not be integrated into a garment. If the faulty sensor electrode was only tested after integration, this could lead to waste of time and resources used to integrate the faulty sensor electrode into a garment only then to find that the component was faulty and could not be used.

Furthermore, pre-fabricated sensor assemblies may be stored for some time before being integrated into garments. It is therefore beneficial to retest the sensor electrode before integration into a garment in order to ensure that the storage has had no adverse effect on the sensor electrode. It may also be possible to detect trends in the data which may indicate an ongoing storage issue. For example, a high failure rate after storage may indicate that the sensor assemblies are being stored at too high a temperature or humidity level. By testing the sensor electrodes and monitoring the trends, it may be possible to identify and remedy such problems.

At each testing stage, information may be written to the memory to indicate that a particular QC/QA step has been performed. The information may comprise a time or date stamp. The information may also comprise information relating to the test performed, including what the test was, where it was performed and by whom. This information may be stored in the memory for later retrieval. It may then be possible to verify at a later time whether or not the QA/QC procedures were correctly followed during manufacture.

Although sensor electrodes have been referred to above, it will of course be appreciated that other components can also be tested in this manner.

A test rig may be used to test the function of a completed smart garment. For example, the integrity of the sensor connections in the garment may be tested. The results of the testing may be written to the memory, for example a programmable memory. The test rig may also write garment information to the memory. The garment information may include a garment type, size, colour, identifier, available sensors, tolerance of electrodes present in the garment, tolerance of sensors present in the garment, an electrical property associated with a component of the garment, manufacturing data and/or an operator ID.

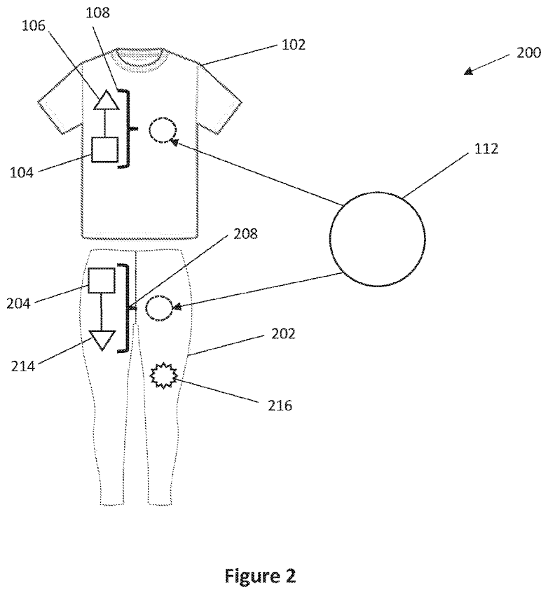

The garment information may be obtained by the test rig scanning a machine-readable code on the garment such as a barcode or a QR code or an AR marker embedded within the fabric, as has been described above with reference to FIG. 2. The data from the test rig may also be sent to a server associated with the garment. When the electronic module is later connected to the garment, it can read the memory and check that the garment was tested successfully (e.g. determine that the appropriate date/time stamp or stamps are present). The electronic module then determines which sensors are available based on the information obtained from the memory. The electronic module may then configure itself to obtain only data for these sensors and transmit the same.

However, if a garment memory is not programmed during manufacture, the memory (e.g. EEPROM) of each garment will have a unique identifier which will allow the memory (and hence the garment) to be uniquely identified. The garment information may be populated in the memory by the electronic module, for example by the electronic module obtaining the relevant information from the server and writing it to the garment memory.

FIG. 2 shows a system 200 comprising an electronic module 112, a first garment 102 and a second garment 202. It will be appreciated that while the first garment 102 is illustrated in the form of a top and the second garment 202 is illustrated in the form of a pair of trousers, these are by way of example only and any type of suitable garment could be used. It will also be appreciated that systems in accordance with the present disclosure may comprise more than two garments.

The first garment 102 is identical to the garment 102 shown in FIG. 1 and will not be described further for the sake of conciseness. The second garment 202 comprises a memory 204, a biosensor 214 and an interface 108 configured to permit the transfer of information between the memory 204 and the electronic module 112 which can be releasably mechanically connected to the interface 208. As indicated by the arrows in FIG. 2, the electronic module 112 can also be releasably mechanically connected to the interface 208 of the first garment 102, as described in more detail above with reference to FIG. 1, such that the electronic module 112 may be used interchangeably with either of the garments 102, 202 of the system 200.

In the system of FIG. 2, the biosensor 214 of the second garment 202 is different to the first biosensor 106 of the first garment 102. It will of course be appreciated that garments may have a plurality of biosensors which may be of the same or different types. Certain garments may have only a subset of available biosensors. As a result, the electronic module 112 may connect to garments having biosensors of different types or garments which do not have a particular biosensor or biosensors which are found in another garment.

In the system 200 shown in FIG. 2, when the electronic module 112 is connected to the interface 108 of the first garment 102, it can receive sensor data from the first biosensor 106 which is of a first type. If the electronic module 112 is then disconnected from the interface 108 of the first garment 102 and connected instead to the interface 208 of the second garment 202, it can receive sensor data from the biosensor 114 which is of a second type. However, if the electronic module 112 is configured to receive signals from a biosensor of the first type, the electronic module 112 may sense only “noise” for the biosensor of the first type which is unavailable on the second garment 202. This noise may then be transmitted back to a server or mobile telephone along with real data for processing. It may be desirable to avoid this occurring in order to save power and bandwidth.

It may be desirable, therefore, for the electronic module 112 to be configured to obtain information from the memory of the garment to which it is connected which allows the electronic module 112 to be correctly configured to receive data from the sensor type present on the garment to which it is connected.

In other words, when the electronic module 112 is connected to the first garment 102 via the interface 108, the electronic module 112 can obtain information from the memory 104 which includes information that enables the electronic module 112 to determine that the first biosensor 106 is present. For example, the information obtained from the memory 104 may be an identifier associated with the first garment 102, a type of the first garment 102, a size of the first garment 102, a colour of the first garment 102, an identifier associated with the first biosensor 106, an electrical property associated with the first biosensor 106, etc. The information received by the electronic module 112 from the memory 104 may allow the electronic module 112 to configure a data stream based on the information obtained from the memory 104. In particular, the electronic module 112 may configure the data stream to transmit data only from the first biosensor 106, for example.

When the electronic module 112 is later disconnected from the interface 108 of the first garment 102, it may then be connected to the interface 208 of the second garment 202. The second garment 202 does not have a biosensor of the same type as the first biosensor 106 of the first garment 102. However, the electronic module 112 may still be configured to receive data from and/or send data to a biosensor of that type such that reconfiguration is required.

The electronic module 112 can obtain information from the memory 204 of the second garment 202 which enables the electronic module 112 to determine that the biosensor 214 is present on the garment 202 and to configure the data stream accordingly, i.e. configure the data stream to transmit only data associated with the biosensor 214. As mentioned above, the information received from the memory 204 may be an identifier associated with the second garment 202, a type of the second garment 202, a size of the second garment 202, a colour of the second garment 202, an identifier associated with the biosensor 214, an electrical property associated with the biosensor 214, etc. It will be appreciated that the information obtained from the memory 204 of the second garment 202 may be of the same or of a different type to the information obtained from the memory 104 of the first garment 102.

The electronic module 112 may comprise submodules which are each configured to interact with (for example, receive data from and/or send signals to) a respective biosensor type. For example, the electronic module 112 may comprise a submodule configured to interact with an ECG sensor and another submodule configured to interact with an EMG sensor. However, as discussed above, some garments may only have a subset of available biosensors. For example, a garment may have an ECG sensor but not an EMG sensor, or vice versa. In this event, it may be desirable for the electronic module 112 to selectively disable or enable at least one of the submodules based on information obtained from a memory of the garment.

Referring again to the system 200 of FIG. 2, the electronic module 112 may have a first submodule configured to interact with the first biosensor 106 and a second submodule configured to interact with the biosensor 214. When the electronic module 112 is connected to the second garment 202 via the interface 208, the electronic module 112 may obtain information from the memory 204 which indicates that the biosensor 214 is present. Based on this information, the electronic module 112 may selectively enable the submodule which is configured to interact with the biosensor 214 and disable the submodule which is configured to interact with the first biosensor 106.

If the electronic module 112 is then disconnected from the interface 208 and instead connected to the interface 108 of the first garment 102, the electronic module may obtain information from the memory 104 that a biosensor of the type of the first biosensor 106 is present. The electronic module 112 may then selectively enable a submodule configured to interact with the first biosensor 106. The electronic module 112 may also receive information from the memory 104 that no biosensor of the same type as biosensor 214 is present. The electronic module may therefore also selectively disable the previously enabled submodule configured to interact with the biosensor 214.

In addition to information identifying the garment and/or the biosensor or biosensors present on the garment, the electronic module 112 may also obtain recorded biodata from the memory. For example, in some implementations, biodata obtained from the biosensors may be stored in the memory of the garment when no electronic module is connected to the garment. Then, at a later point in time, when the electronic module 112 is connected to the garment interface, the biodata may be obtained by the electronic module 112. The electronic module 112 may then transmit the obtained biodata to a server or to another electronic device, for example a mobile telephone. This may further improve comfort for the wearer of the garment as it is not necessary to connect the electronic module to the garment in order for biodata to be collected and stored. As a result, the garment weight can be reduced during wear and the electronic components associated with the smart clothing system can be made to be as unobtrusive as possible during wear.

The garment 202 illustrated in FIG. 2 comprises a machine-readable code 216 which encodes at least a portion of the information stored in the memory 204. It will of course be appreciated that not all garments may be provided with a machine-readable code. The machine-readable code 216 may comprise at least one of: a barcode, a quick-response (QR) code and an augmented-reality (AR) marker. The machine-readable code 216 may be scanned for example by a mobile telephone to allow the mobile telephone to obtain the information encoded within the machine-readable code 216. This may enable the mobile telephone to identify the garment, for example by means of a garment identifier (ID). The mobile telephone may listen out for data transmitted locally (i.e. via wireless transmissions such as via Bluetooth) which includes the garment ID and associated this data with the garment.

The machine-readable code 216 may also be used for motion tracking. In some implementations, the machine-readable code 216 may be a marker may be located on an outside surface of the garment. The at least one marker may comprise a code string identifying the garment encoded into a visual symbol. The marker may be a 2D image. The marker may be a fiducial marker optionally in the form of a 2D image. The marker may be an Augmented Reality (AR) marker with additional information in the form of the code string encoded therein. In some implementations where the machine-readable code 216 is an AR marker, the marker may cause a particular graphic or media to be displayed on a mobile telephone or other electronic device when the mobile telephone or electronic device scans the marker. For example, the marker may be associated with a particular body part and the graphic or media excerpt which is caused to be displayed on the mobile telephone may be associated with the particular body part.

The marker may comprise a plurality of markers. The plurality of markers may be located at different locations on the garment. The plurality of markers may be arranged in a geometric pattern. The plurality of markers may be arranged together on the garment to form a decorative item. The plurality of markers may be located at different locations on the garment. The marker may be integrated into the garment. The marker may be printed onto the garment. Any known garment printing technique may be used such as screen printing or inkjet printing. The marker may be incorporated into the stitching of the garment, and/or a seam of the garment, and/or a hem of the garment, and/or a neckline of the garment, and/or a collar of the garment, and/or a sleeve of the garment, and/or a cuff of the garment, and/or a pocket of the garment, and/or a body of the garment, and/or a fastener of the garment. The fastener may be a zipper, button, clasp, toggle, stud, snap fastener, popper, eyelet, buckle, tie or ribbon.

In some examples, the marker has a limited visual footprint on the garment. This means that the marker is sufficiently small that it is not easily visible by the naked eye but is still visible in the image captured by the image capturing device. In this way, the marker does not affect or has a minimal effect on the appearance of the garment. In some examples, the marker is visible to the naked eye. The marker may be incorporated into or form part of visual element on the garment which may be a decorative item in the garment. The decorative item may be a logo, design, image or pattern on the garment. In this way, the marker may contribute to or enhance the appearance of the garment.

Further aspects of the electronic module will now be described in more detail with reference to FIGS. 3 and 4. FIG. 3 shows a schematic illustration of an electronic module 312. FIG. 4 shows a schematic illustration of another electronic module 412.

Electronic module 312 comprises connections 302 for electrically connecting to an ECG sensor 308 and connections 304 for electrically connecting to an EMG sensor 310. Electronic module 312 also comprises a connection 306 for electrically connecting to an EEPROM memory 314. The connection 306 may be a single-wire bidirectional line (e.g. a one-wire bus) connection. It will be understood by the skilled person that, although only one wire is illustrated in FIG. 3 for connection 306, a one-wire bus connection requires both a data wire and a ground (or return) wire. The electronic module 312 is configured to be releasably mechanically connected to an interface of a garment comprising the memory and sensors.

In some implementations of an electronic module according to the present disclosure, the sensor connections may be common to different biosensors. This enables the electronic module to have fewer external connections and may beneficially ensure that the electronic module can be manufactured to be as small and unobtrusive as possible. Fewer external connections may also lead to lower failure rates and reduced design and manufacturing complexity.

FIG. 4 shows an electronic module 412 having 5 external connections. Three connections 402 are configured to connect to a biosensor 408 provided on a garment. A further two connections 404a, 404b are provided for connection to a memory 406 of a garment. The connections 404a, 404b may be connections of a one-wire bus. For example, connection 404a may be for a data wire and connection 404b may be for a ground wire, or vice versa. It will be appreciated that more or fewer external connections may be provided according to requirements.

Electronic module 412 comprises a plurality of submodules 410a, 410b, 410c, 410d, which may be collectively referred to as submodules 410. It will be appreciated that more or fewer submodules 410 may be provided. The submodules 410 are each configured to interact with a particular type of sensor which may be present on a garment. For example, submodule 410a may be configured to interact with an ECG sensor, submodule 410b may be configured to interact with an EMG sensor, submodule 410c may be configured to interact with an IMU sensor and submodule 410d may be configured to interact with a MEG sensor.

The electronic module 412 may be releasably connected to all manner of garments. Not all of the garments will comprise the same sensors. Some garments may comprise only one biosensor; other may comprise a plurality of biosensors. It is desirable for the electronic module 412 to be able to operate with all kinds of sensors or at least as many sensors as possible.

In some implementations, the electronic module 412 may comprise as many submodules 410 as are required to ensure compatibility with all of the kinds of biosensors which may be present in a garment to which the electronic module 412 may be connected. The electronic module 412 may be configured to selectively enable at least one of the submodules 410 in order to ensure that submodules which are relevant to the sensors present on the garment to which the electronic module 412 is connected are active. The electronic module 412 may also be configured to selectively disable at least one of the submodules 410 if it is determined that the particular submodule to be disabled is not relevant to any of the sensors present on the particular garment to which the electronic module 412 is connected. In this way, it may be possible to conserve power. In addition, the electronic module 412 may configure a data stream such that only information relating to the appropriate sensor is transmitted as part of the data stream. In this way, it is possible to avoid noise from the other sensors and/or submodules polluting the data stream. In addition, the data stream may use less bandwidth and the transmission may require less power.

In an example, the electronic module 412 may obtain information from the memory 406 of the garment to which the electronic module 412 is connected that indicates that the biosensor 408 is an IMU sensor. The electronic module 412 may then selectively enable the submodule 410c based on the information obtained from the memory 406, since the submodule 410c is the relevant submodule for interacting with an IMU sensor. The electronic module 412 may also selectively disable submodules 410a, 410b and 410d since these are not required for interaction with an IMU sensor. In this way, the electronic module 412 may conserve power.

It will of course be appreciated that multiple submodules may be active at one time. For example, where a garment comprises a plurality of sensors, the electronic module 412 may selectively enable the corresponding plurality of submodules.

In some implementations, the same submodule may be configured to operate with different sensors. For example, the submodule 410a may be capable of operating with both an EMG sensor and an ECG sensor, depending on how the submodule 410a is configured. If the electronic module 412 is connected to a garment having an EMG sensor, the electronic module 412 may determine that an EMG sensor is present based on the information obtained from the memory. The electronic module 412 may then configure the submodule 410a in such a way that information can be transmitted between the EMG sensor and the submodule 410a or the electronic module 412. For example, the submodule 410a may be driven in a way which is compatible with data obtained from the EMG sensor. If the electronic module 412 is then connected to a garment which comprises an ECG sensor, the electronic module 412 may determine than an ECG sensor is present and reconfigure the submodule 410a to operate with the ECG sensor. For example, the submodule 410a may be driven in a different way in order to be compatible with the ECG sensor.

Referring now to FIG. 5, various advantages associated with a single-wire bidirectional line (e.g. a one-wire bus) will be discussed. FIG. 5 schematically illustrates single-wire bidirectional line 504 for connecting an EEPROM memory 506 to an interface 502 of a garment for connection to an electronic module as disclosed herein. Also connected to the single-wire bidirectional line are a humidity sensor 508 and a temperature sensor 510. The EEPROM memory 506, the humidity sensor 508 and the temperature sensor 510 each have a single-wire input-output interface for connection to the single-wire bidirectional line. It will be appreciated that this is by way of example only and that other sensors may alternatively or additionally be connected to the single-wire bidirectional line 504. It will further be appreciated that the memory need not be an EEPROM memory but may be any other suitable form of memory known to the skilled person.

In some implementations, temperature sensors may be included in the electronic module. Such sensors approximate the temperature of the skin by measuring the temperature of a processor within the electronic module and/or the temperature of other electronic components or a dedicated temperature chip within the electronic module and using an algorithm which converts the processor or component temperature to the skin temperature. This relationship is determined using a calibration procedure using a temperature sensor in contact with the skin as a reference. However, this is a less accurate method of measuring skin temperature than placing a temperature sensor in contact with the skin.

In other implementations, a temperature sensor may be provided in the garment itself which can be placed next to the skin to obtain a direct reading of skin temperature. This may be more accurate than measuring an indirect processor temperature. Likewise, the garment may have a humidity sensor which is arranged to be placed next to the skin.

It may be desirable for the skin temperature reading to be obtained as far away from the electronic module as possible to avoid any heat generated by the electronic module from influencing the temperature reading. As a result, if the electronic module is positioned on the right-hand side of the garment, the temperature sensor 510 may be positioned on the left-hand side, or vice versa. Similarly, if the electronic module is positioned toward the bottom of the garment, the temperature sensor 510 may be positioned toward the top of the garment, or vice versa.

A benefit of having the temperature sensor and/or humidity sensor of the garment connected to the one-wire bus 504 is that if it is not possible to obtain information from the memory, it can be assumed that there is a fault on the one-wire bus. The temperature and/or humidity readings can then be disregarded.

Owing to the construction of the single-wire bidirectional line, it is likely that any fault is the result of a break in connection rather than a short. For example, even if the data wire and ground wire for the one-wire bus are tracked together, a pitch spacing of 1.5″ may be required owing to loom tolerance. In this case, the data wire and ground wire will be spaced far enough apart that contact between the two wires is unlikely, even if the traces follow substantially the same path, such that a short circuit should not be formed. In the event of a fault, it is much more likely that a connection has failed owing to breakage.

In the example illustrated in FIG. 5, it may be possible to determine where a break in connection of the single-wire bidirectional line 504 has occurred based on the data which can be obtained from the EEPROM 506 and the sensors 508, 510. For example, if it is possible to obtain information from the EEPROM 506 and the humidity sensor 508 but not from the temperature sensor 510, it can be assumed that any fault in the one-wire bus 504 lies in the section which connects the humidity sensor 508 and the temperature sensor 510. This information may assist with troubleshooting and repair.

At least some of the example embodiments described herein may be constructed, partially or wholly, using dedicated special-purpose hardware. Terms such as ‘component’, ‘module’ or ‘unit’ used herein may include, but are not limited to, a hardware device, such as circuitry in the form of discrete or integrated components, a Field Programmable Gate Array (FPGA) or Application Specific Integrated Circuit (ASIC), which performs certain tasks or provides the associated functionality. In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. An electronic module configured to be releasably mechanically connected to an interface of a wearable article, wherein the electronic module is configured to obtain wearable article size information from a memory of the wearable article, and is further configured to a determine a compensation that should be performed to sensor data received from the wearable article to compensate for electrical properties of the wearable article using the wearable article size information.

2. The electronic module as claimed in claim 1, wherein the electronic module is further configured to obtain information relating to the electrical properties of the wearable article from the memory of the wearable article, and is further configured to determine a compensation that should be performed to sensor data received from the wearable article to compensate for electrical properties of the wearable article using the wearable article size information and the information relating to the electrical properties.

3. The electronic module as claimed in claim 2, wherein the information relating to the electrical properties identifies the impedance of one or more electrodes of the wearable article.

4. The electronic module as claimed in claim 2, wherein the information relating to the electrical properties comprises calibration information obtained as a result of one or more testing functions performed on the wearable article.

5. The electronic module as claimed in claim 1, further configured to write information to the memory of the wearable article when the electronic module is connected to the interface of the wearable article.

6. The electronic module as claimed in claim 1, wherein the electronic module is configured to configure a data stream based on information obtained from the memory.

7. The electronic module as claimed in claim 1, wherein the electronic module comprises a plurality of submodules and wherein the electronic module is configured to selectively enable and/or disable at least one submodule based on information obtained from the memory.

8. The electronic module as claimed in claim 1, wherein the electronic module is configured to transmit information to a server and/or to write information from the server to the memory.

9. The electronic module as claimed in claim 1, further comprising a single-wire input-output interface for connection to the memory associated with the wearable article.

10. The electronic module as claimed in claim 1, wherein the electronic module is configured to perform a testing function on the wearable article and write a result of the testing function to the memory of the wearable article.

11. The electronic module as claimed in claim 1, wherein the electronic module is configured to write usage information to the memory.

12. The electronic module as claimed in claim 11, wherein the electronic module is configured to increment a counter in the memory when the electronic module is connected to the interface.

13. A wearable article comprising:
a first biosensor;
a memory configured to store wearable article size information; and
an interface for releasable mechanical connection to an electronic module, wherein the interface is configured to permit the transfer of the wearable article size information from the memory to an electronic module connected to the interface.

14. The wearable article as claimed in claim 13, wherein the memory is further configured to store information relating to the electrical properties of the wearable article.

15. The wearable article as claimed in claim 14, wherein the information relating to the electrical properties identifies the impedance of one or more electrodes of the wearable article.

16. The wearable article as claimed in claim 13, wherein the interface is further configured to permit the transfer of information between the first biosensor and the electronic module when the electronic module is connected to the interface.

17. The wearable article as claimed in claim 13, wherein the memory is configured to store information relating to how many times an electronic module has been connected to the interface.

18. The wearable article as claimed in claim 17, wherein the memory comprises a counter configured to be incremented each time an electronic module is connected to the interface.

19.-21. (canceled)

22. The wearable article as claimed in claim 13, wherein the wearable article comprises a machine-readable code, wherein the machine-readable code encodes at least a portion of the information stored in the memory.

23.-24. (canceled)

25. A method of operating an electronic module, the method comprising:
connecting the electronic module to an interface of a wearable article;
obtaining, by the electronic module, wearable article size information from a memory of the wearable article; and
determining a compensation that should be performed to sensor data received from the wearable article to compensate for electrical properties of the wearable article using the wearable article size information.
